(19) United States
(12) Plant Patent Application Publication
(10) Pub. No.: US 2003/0009802 P1
(43) Pub. Date: Jan. 9, 2003

(54) Chrysanthemum plant named 'Tifata'

(75) Inventor: Robert Noodelijk, Woubrugge (NL)

Correspondence Address: PARKHURST & WENDEL, L.L.P., 1421 PRINCE STREET, SUITE 210, ALEXANDRIA, VA, 22314-2805, US

(73) Assignee: Chrysanthemum Breeders Association N.V., Willemstad (AN)

(21) Appl. No.: 10/175,869

(22) Filed: Jun. 21, 2002

(30) Foreign Application Priority Data
Jun. 28, 2001 (NL) CHR3272

(51) Int. Cl. A01H 5/00

(52) U.S. Cl. PLT/286

(57) ABSTRACT

A Chrysanthemum plant named ‘TIFATA’ characterized by its medium sized blooms with red-yellow ray-florets and yellow-green disc florets.

### BACKGROUND OF THE INVENTION

‘TIFATA’ is a product of a breeding-program which had the objective of creating new chrysanthemum cultivars with a daisy type flower, a 7 week response and a medium plant height. The new plant of the present invention comprises a new and distinct cultivar of Chrysanthemum plant. ‘TIFATA’ is a seedling from a cross in a breeding program maintained under the control of inventor. The female parent is 94.1301, the male parent is 94.1106, both unpatented, unamed seedlings. The parents are not available to inventor for description. The new and distinct cultivar was discovered and selected as a flowering plant within the progeny of the stated cross by Rob Noodelijk in a controlled environment (greenhouse) in Rijsenhout Holland in April 1997. The first act of asexual reproduction of ‘TIFATA’ was accomplished when vegetative cuttings were taken from the initial selection in June 1997 in a controlled environment in Rijsenhout Holland.

### SUMMARY OF THE INVENTION

The present invention is a new and distinct variety of chrysanthemum bearing medium sized blooms with red-yellow ray-florets and yellow-green disc florets.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

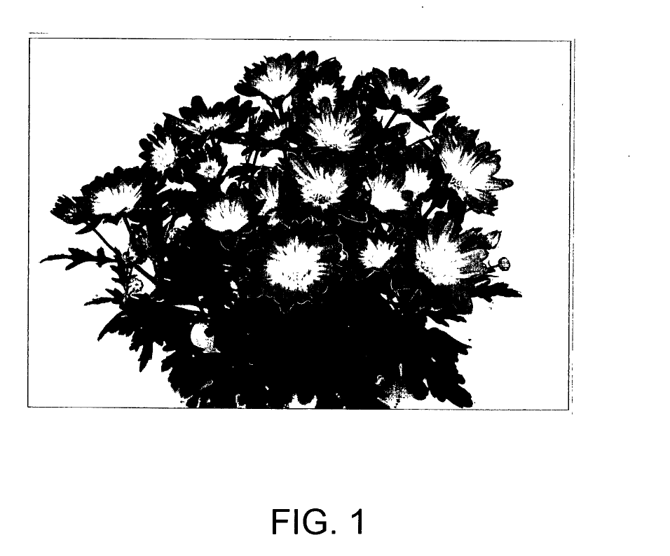

FIG. 2 shows the various stages of bloom of the new cultivar.

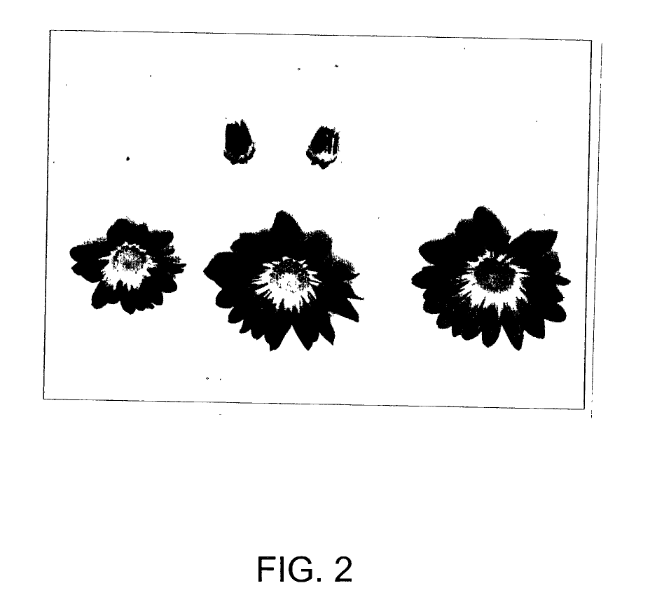

FIG. 3 shows the foliage of the new cultivar

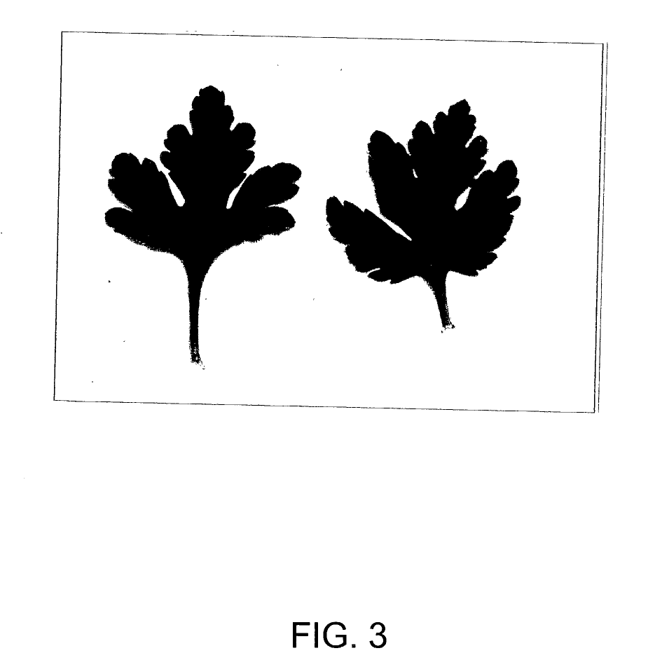

### DESCRIPTION OF THE INVENTION

This new variety of chrysanthemum is of the botanical classification chrysanthemum morifolium. The observations and measurements were gathered from plants grown in a greenhouse in Rijsenhout Holland in a photo-periodic controlled crop under conditions generally used in commercial practice. The greenhouse temperatures during this crop were at day-time between 18° C. and 25° C. and at night 20° C. The photo-periodic response time in this crop was 48 days after an average of eight long days. After this long day period to flowering growth retardants were applied 6 times in an average dose of 1.5 gram/liter water. The plants were observed (directly) during the flowering of this crop. No tests were done on disease or insect resistance or susceptibility. No tests were done on cold or drought tolerance. This new variety produces medium sized blooms with red-yellow ray-florets and yellow-green disc-florets blooming on the plant for 5 weeks. This new variety of chrysanthemum has been found to retain its distinctive characteristics throughout successive propagations however the phenotype may vary significantly with variations in environment such as light intensity and temperature. To show the phenotype as described ‘TIFATA’ can be planted without assimilation lightning (high pressure sodium lamps) between week 50 and week 40 of the next year under greenhouse conditions in Holland. With assimilation lightning (minimum level 2500 lux) it can be planted year round under greenhouse conditions in Holland.

From the cultivars known to inventor the most similar existing cultivar in comparison to ‘TIFATA’ is ‘Bajimba’ (Pat.Pend). When ‘Bajimba’ and ‘TIFATA’ are being compared the following differences are noticed: The difference of ‘Bajimba’ and ‘TIFATA’ are (1) flower size and (2) flower color.

The following is a description of the plant and characteristics that distinguish ‘TIFATA’ as a new and distinct variety. The color designations are taken from the plant itself. Accordingly, any discrepancies between the color designations and the colors depicted in the photographs are due to photographic tolerances. The color chart used in this description is: The Royal Horticultural Society color chart, edition 1995.

Botanical Description of CULTIVAR ‘TIFATA’

Bud

Size.—Small;cross-section 1.0 cm, height 0.8 cm

Outside color.—Yellow-orange 20 C at the base, orange-red 34 B

Involucral bracts.—2 rows, length 7 mm, width 3 mm

Involucral bracts among disc-florets.—Not present

Involucral bracts color.—Yellow-green 144 A

Bloom

Type.—Daisy

Height.—Flat

Size.—Medium

Fully expanded.—6.5 cm

Number of blooms per branch.—Approx. 3-5 blooms per branch

Performance on the plant.—5 weeks

Seeds.—Produced in large quantities, oval shaped, grey-brown 199A, 1.5 mm in length.

Fragrance.—Typical chrysanthemum

Color

Center of the flower (disc-florets).—Immature yellow-green 153 D, yellow-green 144 C in the center Mature yellow 12 A

Color of upper surface of the ray-florets.—Yellow 5 A at the base, greyed-red 179 A

Color of the lower surface of the ray-florets.—Yellow 4 A at the base and in longitudinal stripes, greyed-red 173 B in between and at the edge

Tonality from Distance.—A pot mum with red-yellow flowers and a yellow-green disc

Color of the surface of the ray-florets after aging of the plant.—Yellow 5 A with greyed-red 173 B

Ray florets

Texture.—Upper and under side smooth

Number.—22

Cross-section.—Flat to convex

Longitudinal axis of majority.—Reflexing; twisted when mature

Length of corolla tube.—Short

Ray-floret length.—3.0-3.2 cm

Ray-floret width.—1.5 cm

Ratio length/width.—Very low

Shape of tip.—Round

Disc florets

Disc diameter.—1.6 cm

Distribution of disc florets.—Numerous, clearly visible at all stages of flowering

Shape.—Tubular

Color.—Yellow-green

Receptacle shape.—Conical raised

Reproductive Organs

Stamen (present in disc florets only).—Thick, 4 mm in length

Number of stamen.—4

Stamen color.—Yellow-green

Pollen.—Appears soon

Pollen color.—Yellow 12 A

Styles (present in both ray and disc florets).—Thick

Style color.—Yellow-green

Style length. 6 mm

Stigmas.—Yellow-green

Stigma width.—2 mm

Ovaries.—Enclosed in calyx

Plant

Form.—A pot mum meant for indoor use

Growth habit.—Spreading

Growth rate.—Moderate

Height.—24.0-26.0 cm

Width.—26.0 cm

Stem color.—Yellow-green 144 A

Stem strength.—Strong

Stem brittleness.—Absent

Stem anthocyanin coloration.—Absent

Length of lateral branch.—Lower branches 14.0-15.0 cm, upper branches 12.0-13.0 cm

Lateral branch color.—Yellow-green 144 A

Lateral branch, attachment.—Strong

Branching (average number of lateral branches).—Prolific with 5-7 breaks after pinching

Peduncle length.—4.0-4.5 cm

Peduncle color.—Yellow-green 144 A

Flowering Response(photo-periodic controlled crop, not natural season.—48 Days

Foliage

Color mature.—Upper side green 137 A Under side green 138 B

Color immature.—Upper side green 137 A Under side green 138 B

Size.—Small; length 6.5 cm, width 5.5 cm

Quantity (number per lateral branch).—6-7

Shape.—Round and lobed

Texture upper side.—Glabrous

Texture under side.—Pubescent

Venation arrangement.—Palmate

Shape of the margin.—Crenated

Shape of base of sinus between lateral lobes.—Round

Margin of sinus between lateral lobes.—Converging

Shape of base.—Truncate, sometimes cordate

Apex.—Mucronate

Differences with the comparison varieties When grown under the same conditions

| | ‘TIFATA’ | ‘Bajimba’ |
|---|---|---|
| Flower size | 6.5 cm | 7.5-8.0 cm |
| Flower color | Greyed-red 179A, yellow | Red 46A, yellow 5B at the |
| | 5A at the base till half | base and in |
| | of the ray-florets. | longitudinal stripes |

I claim:

1. A new and distinct variety of chrysanthemum plant as described and illustrated.
